(19) United States
(12) Patent Application Publication
(10) Pub. No.: US 2022/0344006 A1
(43) Pub. Date: Oct. 27, 2022

(54) COMPUTER IMPLEMENTED METHOD TO OPTIMIZE PHYSICAL-CHEMICAL PROPERTIES OF BIOLOGICAL SEQUENCES

(71) Applicants: POLITECNICO DI TORINO, Torino (IT); CENTRO DE INMUNOLOGÍA MOLECULAR, La Habana (CU)

(72) Inventors: Guido UGUZZONI, Torino (IT); Andrea PAGNANI, Torino (IT); Jorge FERNANDEZ DE COSSIO DIA, La Habana (CU)

(73) Assignees: POLITECNICO DI TORINO, Torino (IT); CENTRO DE INMUNOLOGÍA MOLECULAR, La Habana (CU)

(21) Appl. No.: 17/620,768

(22) PCT Filed: Jun. 19, 2020
(86) PCT No.: PCT/IB2020/055780

§ 371 Date: Dec. 20, 2021

(30) Foreign Application Priority Data
Jun. 19, 2019 (IT) 102019000009531

(51) Int. Cl. G16B 35/20; G06F 17/11

(52) U.S. Cl. CPC G16B 35/20; G06F 17/11

(57) ABSTRACT

A computer based biological sequence analysis method provides, after a training phase adopting data from screening experiments, either an evaluation of an input sequence expressing the performance with reference to the chemical-physical feature object of the screening experiment, or at least an optimized output sequence. The method provides the use of a set or library of sequences derived from DMS experiments and SELEX for the generation of a second set of high efficiency biological sequences, whereby high efficiency means, for example, high catalysis capacity, high fitness, high ability to bind to a specific target, high fluorescence activity and, in general, a high performance with reference to the chemical-physical properties of a molecule which are defined at the start and can be selected through experiments.

### CROSS REFERENCE TO THE RELATED APPLICATIONS

This application is the national phase entry of International Application No. PCT/IB2020/055780, filed on Jun. 19, 2020, which is based upon and claims priority to Italian Patent Application No. 102019000009531, filed on Jun. 19, 2019, the entire contents of which are incorporated herein by reference.

### TECHNICAL FIELD

The invention relates to a computer implemented method for optimizing the physical-chemical properties of polymeric biological molecules, in particular proteins and nucleic acids. In particular, the invention relates to an automatic computational method for predicting, identifying, selecting and generating sequences with optimal characteristics in relation to a physical-chemical property of interest of a molecule.

In particular, the invention relates to a method of analysis and selection of data derived from Deep Mutational Scanning (DMS) and Direct Evolution (DE) experiments.

The invention also relates to a method for predicting the physical and chemical properties of biological sequences based on the use of data deriving from HTS-SELEX (High-Throughput Sequencing SELEX) experiments.

### BACKGROUND

The Deep Mutational Scan and Direct Evolution methods are approaches to study the impact of different protein variants obtained by mutagenesis and prepare a functional selection of these variants.

Being able to generate an amino acid sequence and at the same time predict its specific behavior remains an unresolved challenge, in particular for all those industrial and pharmaceutical applications that need reliable products and high yields.

Currently, approaches to the engineering of proteins in a laboratory fall into two main categories: in-vivo experimental methods, such as guided evolution, or in-silico methods such as, for example, rational design.

In the first case, the biological evolutionary process is reproduced and accelerated in the laboratory (Packer and Liu—Nature Reviews Genetics—2015); the technique involves the creation of a library of mutants of the reference protein and the execution of progressive cycles including a selection step in which the mutated variants are expressed and isolated for a given function, and a step of further mutagenesis of the selected mutants. This technique has been successfully applied in optimizing enzymes for chemical synthesis. The guided evolution technique has the fundamental limitation of being able to explore only a small fraction of the enormous amount of theoretically possible variants of a given sequence. For this reason, normally the samples that are tested are those that have either well-defined point mutations or mutants generated by recombining existing and known sequences.

The rational design methods instead are based on structural information initially deriving from crystallization experiments or from in-silico predictions of possible structures of a protein; simulations of possible protein structures and sequences are therefore carried out using estimates relating to changes in free energy, due to amino acid substitutions, which can be calculated using score functions.

For this approach, efficient algorithms are needed in conformational research, often with libraries of rotamers for the side chains, in order to exemplify the possible conformations of the designed or modified structures. Normally scoring functions and conformational search algorithms use native protein information or an interface known as the starting point.

There are also approaches attempting to generate functional proteins using a sequence-based strategy. This approach uses machine learning techniques to describe statistical constraints of homologous sequences to a specific target protein (Barrat-Charlaix et al.—Scientific reports) (Hopf et al.—Nature Biotechnology). There are, however, state of the art few cases in which it has been possible to successfully design a de-novo protein. Bakers and colleagues (Po-Ssu Huang et al. Nature 2016) managed to design and create proteins with folding structures never described in nature and to alter the protein-protein interfaces.

Deep Mutational Scanning (DMS) is a recent approach that combines the selection of data from large-scale mutagenesis experiments to investigate the protein function and high-throughput sequencing of DNA sequencing techniques in order to quantify the activity of different variants of a protein (Fowler D M and Fields S.—Nat Methods, 2014).

In this type of approach, a library of protein variants is initially introduced into a model organism, for example a phage, a bacterium, a yeast or a mammalian cell culture. Once the protein of interest and its different mutants are expressed, a selection step is performed for the expected protein function or for other molecular properties of interest; this step has the function of enriching the frequency of each variant based on its functional capacity, for example fitness, the ability to bind to a selected target or to catalyze an enzymatic reaction.

At each cycle, the frequency of each mutant is determined using DNA deep sequencing techniques to calculate the number of times each variant appears. The advent of these sequencing techniques has greatly increased the ability to test the sequence-function relationship of proteins.

To date, the analysis of DMS data is mainly based on the enrichment score attributed to each protein variant; this score is used to quantify the propensity of a mutant to be selected and is calculated as the ratio of the frequencies of the mutants before and after the selection, and compared with the score of the protein of origin (wild-type) at each round.

The reference software is Enrich2 (Alan F. Rubin et al. Genome Biology 2017). This software uses measures to correct the score based on sampling errors and consistency with experiment replicas, when available. In the case of multiple cycles, it uses a linear regression to evaluate the final score.

Despite the measures used, the enrichment score is affected by statistical noise of various nature, for example the noise due to sampling, the low reproducibility for replicates of the experiment and the non-linearity of the variation of frequencies in the time series, and the strong dependence of the same from the initial library and other features of the experiment.

Another tool for analyzing data from DMS is dms_tools2 (Jesse D Bloom BMC Bioinformatics 2015). Other publications of interest in the analysis of DMS data are the following:

Otwinowski, Jakub. “Biophysical inference of epistasis and the effects of mutations on protein stability and function.” Molecular biology and evolution 35.10 (2018): 2345-2354.

Aptamers are short RNA or DNA molecules, capable of binding to target molecules with high affinity and specificity, with great potential for application in therapy and in the diagnostic field. One of the most used techniques for the selection of functional aptamers is called SELEX (Systematic Evolution of Lipids by Exponential Enrichment).

The classic method involves the synthesis of a library of unique oligonucleotide sequences, having a structure with a central portion that contains a randomly generated sequence side-by-side with two end portions of the preserved sequence. The steps of this technique involve several selection cycles for binding to target molecules, separation of the selected sequences and subsequent amplification.

At the end of several cycles, the sequences with higher binding capacity will be enriched and their frequency will be determined using deep sequencing techniques. In this case we speak of High-Throughput Sequencing SELEX (HTS-SELEX). There are several derivations of this technique that however maintain the concept of selection and enrichment of the most functional sequences.

Just as for DMS approaches, the manipulation of a very large amount of data is required and there are different methods of analysis for the study of the enrichment of the sequences.

The need therefore remains to develop an effective and reliable method for the in-silico analysis of sequence libraries obtained from screening experiments (DMS, DE and SELEX) that allows the selection of mutants with the desired characteristics, the prediction of the fitness of mutants not experimentally tested and the generation of mutants with optimized characteristics. In particular, there is a need for a method for predicting the effects of mutations that takes into account the non-additive epistatic effects of individual mutations.

Shen et Al. in ‘Protein engineering, design and selection, vol. 21 no. 1 of 19 Dec. 2007 describes a method in which the input data for model training require experimentally measured values of Gibbs free energy, temperature and pH at which Gibbs free energy is measured. This requires significant investments in time and costs to carry out the measurements.

### SUMMARY

The problems of prior art have been solved with the method according to the present invention. The inventors have now developed a method for analyzing data libraries derived from DMS, DE and SELEX experiments.

Therefore, the object of the invention is to provide an effective and reliable method for in-silico screening of sequence libraries obtained from such experiments and for selecting the best mutants for the desired characteristic.

A further object of the invention is to provide the use of a set or library of sequences derived from DMS experiments and SELEX for the generation of a second set of high efficiency biological sequences, whereby high efficiency means, for example, high catalysis capacity, high fitness, high ability to bind to a specific target, high fluorescence activity and, in general, a high performance with reference to the chemical-physical properties of a molecule which are defined at the start and can be selected through the experiments indicated above.

The scopes of the present invention are at least partially achieved by a computer based method to analyze biological sequences to optimize biophysical properties of biological sequences (e.g., Proteins, RNA, DNA), related to a chemical-physical characteristic of the molecule, which also includes a biological characteristic, such as binding to a specific target molecule (e.g., an antibody that binds a specific antigen), catalytic effect, fluorescence, thermostability, immunization power (IC50 in the activity of antibodies against infections). The method comprises the steps of:
Defining the molecular states allowed or of interest (e.g., a macroscopic state of the molecule as bound, unbound, folded, unfolded) relating to the activity or physical-chemical characteristic of interest of the molecule.
Defining at least one function, hereinafter defined statistical energy, associated with the allowed molecular states. These statistical energies of the different states are functions of a generic sequence and therefore define a mapping from genotypes to phenotypes (i.e., a map from the sequence to the physical-chemical activity considered). These functions depend on a multivariate linear function on scalar parameters that can be related to position bias, for example the parameters that contain the energy contribution of an amino acid in a specific position of the protein, and to epistatic effects such as interactions in pairs and higher level (for example triplets, quadruplets), which take into account the non-additivity of the mutation effects.

Providing a likelihood function describing one or more rounds of a screening experiment (e.g., Directed Evolution, Deep Mutational Scan, SELEX). This probability function comprises at least a first probabilistic factor expressing the probability that a sequence is selected on the basis of at least one statistical energy function. Preferably other factors are also present, for example a second probabilistic factor expressing the probability that a given sequence is amplified during the experimental screening process. More generally, the probabilistic factors of the likelihood function are each a probabilistic expression of a step of the screening experiment. In particular, these probabilities are dependent: a) on the model parameters through the above definitions of statistical energy functions for each molecular state b) on the number of samples for each variant detected by the sequencing of the experimental screening rounds performed before applying this method.

Determining the parameters of at least one statistical energy function by maximizing the posterior probability of the parameters themselves, being assigned the training data from the sequencing of one or more of the screening experiments selecting the variants of the molecule for the desired physical-chemical characteristic (e.g., linking with different targets). In particular, the parameters for each possible sequence variant are calculated.
Calculating the statistical energy function associated with the molecular states of the sequence considered, based on the parameters obtained through the training phase.
Providing a score to an assigned sequence relating to the desired physical-chemical characteristic based on the at least one statistical energy function. For example, to obtain a prediction of affinity with a target of an assigned sequence, the score is defined by means of the statistical energy relating to the sequence for the state linked to the target. Since the parameters for each possible sequence variant are available, the statistical energy is calculated on the basis of the assigned sequence. Different sequences assigned after the calculation of the energy parameters will correspond to different score values.
Generating a sequence or a sequence library with a score maximized or higher than a predefined threshold, on the whole set of possible sequences or for a specific subset of it. This set should preferably be understood as the set of sequences of length equal to the sequences of the experiment used for training. According to this use of the energy parameters, an optimization algorithm is applied which finds the sequence or sequences capable of maximizing the energy parameters or maximizing a function of the energy parameters already calculated during the training phase. Through the method, in particular through the attribution of the score, it is possible to process a large number of input sequences to analyze the behavior of the mutants with respect to the desired chemical-physical characteristic, e.g., catalysis, fitness, ability to bind to a specific target, fluorescence activity, immunization power.

It should be noted that the desired chemical-physical characteristic is identified through the set of biological sample sequences used to train the model, i.e., to calculate the parameters of the multivariate linear function. This set comprises a plurality of sequences that are equipped/exhibit/are effective with respect to the target chemical-physical characteristic.

In this way, it is possible to estimate in-silico via the score, for each sequence assigned and processed by the method, how much this input sequence is related, that is, it is provided or exhibits or is effective with respect to the target chemical-physical property.

Furthermore, a sequence library with high affinity to the target chemical-physical property includes a number of potentially effective protein sequences to be used in successive experimental phases in vitro or in vivo.

It should also be noted that the parameters of the multivariate linear function are calculated on the basis of input sample sequences during a training phase. The use of sample sequences as input data, preferably only type of input data, of the training phase, to obtain unsupervised training, allows a considerable saving since specific measures of parameters such as pH and/are not necessary or the Gibbs temperature and/or free energy. According to the method of the invention, Gibbs free energy is calculated or calculable through the method and is not measured in the experiments that define the input data.

It has been verified that it is possible to formulate a likelihood function of a generic screening experiment and that a relative factor representing the selection phase, always present in such experiments, can be expressed on the basis of one or more multivariate and linear statistical energy functions, each having parameters referring to all possible sequences with the devices described in the examples discussed below. A skilled man is able, on the basis of the phases of a specific screening experiment, to formulate any probabilistic factors additional to that of selection and suitable for completing the likelihood function.

The calculation of the scalar parameters of the at least one statistical energy function by maximizing the likelihood function allows the latter to be used as the basis for calculating a score for a generic sequence, which can have various expressions and, in particular, the simplest one in which the score of a sequence is the sum of the scalar parameters relating to said sequence.

The parameters of the statistical energy function or functions, after being calculated, can therefore be used both to evaluate one or more input sequences by means of a score, or to generate one or more sequences that maximize a function of the energy parameters.

In addition, it is possible to:
provide a score that considers different biophysical properties of the molecule, based on the statistical energy functions associated with each property of interest; for example, it is possible to define a score that takes into account both the stability and the affinity of the molecule by combining the binding energy with that of folding; and/or
provide a score that uses multiple screening experiments of the same molecule by selecting different functions (for example binding with different target molecules); in this case, the method provides sequence energies related to each experiment (i.e., the different functions) and it is possible to combine these energies in personalized scores for a desired combination of biophysical functions of the molecule; for example, this is useful to design multi-specific variants with binding energies assigned to the targets used in the screening (for example bispecific monoclonal antibodies or more generally multispecific antibodies).

In one embodiment of the method, the optimized library can be used as a set of initial sequences for subsequent experiments or for the generation of new randomized sequences. This process is to be understood in a pipeline comprising cycles of screening experiments and in-silica training of the model and generation of mutant libraries through it.

Other purposes will be evident from the detailed description of the invention, which follows.

### BRIEF DESCRIPTION OF THE DRAWINGS

FIG. 1 shows a general flowchart related to an example of input/output of this method.

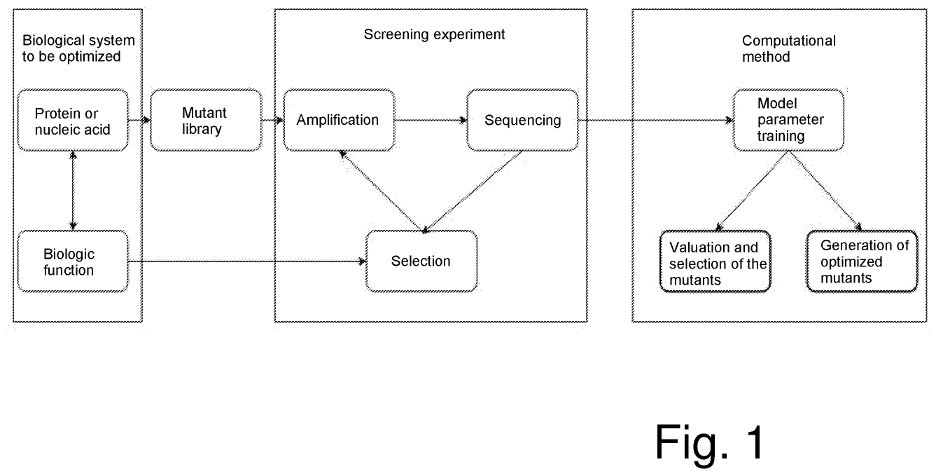

FIG. 2 shows a flow chart related to an example of a pipeline in which this method can be found: starting with the pre-processing of raw data from the sequencing of the experiment to three examples of model output, not to be considered exhaustive of its uses.

FIG. 3 is a schematic representation of the main definitions used in the description of the model.

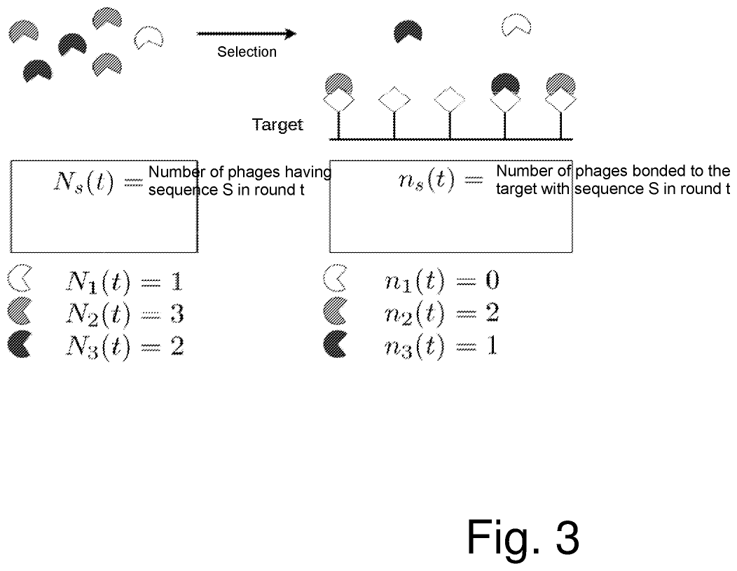

Left panel: Nₛ(t) is the number of vectors (e.g., phages) exposing sequence s. The number of reads obtained from sequencing is proportional to Nₛ(t).

Right panel: nₛ(t) is the number of vectors with sequence s that have been selected (for example that have bonded with the target).

FIG. 4 shows the assessment of the selectivity of mutants. Scatter plot of the statistical binding energy calculated by the model and the selectivity calculated on the data (selectivity formula indicated in the following paragraphs). The four panels in FIG. 4 are related to the experiments described in example 1.

The circles (crosses) are the sequences on the test set (training set) with an initial count beyond a certain threshold (procedure for checking data quality). In each panel there is the Spearman correlation coefficient for each case. The Spearman coefficient measures the degree of ordinal relationship between two variables. The strong correlation, value of the coefficient between 0.80 and 0.98, shows the model's ability to predict the binding affinity in each of the four experimental datasets.

FIG. 5 shows the assessment of the selectivity of mutants through the binding energy dispersion graph calculated by the model and the selectivity calculated on the data.

FIG. 5 shows the evaluation of the selectivity of the sequences of an experiment (example 2) by learning the model on another experiment related to the same protein (example 3).

The points therefore correspond to sequences belonging to experiment 2 with a total count beyond a certain threshold (procedure for checking data quality). The selectivity calculated from the counts in experiment 2 is shown on the abscissas and the statistical energy of the model initialized by the data of experiment 3 is indicated on the ordinates.

The strong correlation, Pearson's coefficient, shows the model's ability to predict binding affinity in an experiment independent of that of learning. The Pearson coefficient between two statistical variables expresses the extent of any linearity relationship between them.

FIG. 6 shows the assessment of the selectivity of mutants through the scatter plot of bond energy calculated by the model and selectivity calculated on the data.

FIG. 6 shows the evaluation of the selectivity of the sequences with high selectivity (in this case high binding affinity) by learning the model on low selectivity sequences. The data relates to experiment 1. It should be noted that in this test the sequences with high affinity and therefore a priori more informative are hidden from the model during the learning or initialization phase.

The black dots correspond to the low selectivity training sequences while the gray crosses correspond to the high selectivity test sequences.

The model score correctly sorts the sequences in relation to the experimental selectivity.

### DETAILED DESCRIPTION OF THE EMBODIMENTS

The definitions listed below are used in the description of the invention.

By Deep Mutational Scanning (DMS) a methodology is meant that is based on next generation sequencing techniques to measure the activity of a number of unique variants in the order of 105 (or more) in a single experiment a protein, a DNA sequence or a reference RNA.

By Selex (Systematic evolution of ligands by exponential enrichment) a combinatorial biochemical technique is meant that is suitable for the production of DNA or RNA oligonucleotides (both single and double chain) capable of specifically binding a given target called aptamer.

By Directed Evolution or DE, a technique is meant in which a library of variants is built from one or more sequences and is subjected to a selection process for a property of interest, the best variant or a selection of variants is used for the next round in which the procedure is repeated.

Machine-learning-assisted Directed Evolution means a Direct Evolution process in which an in silico model is trained starting from the sequence selection data (sequencing of a sample of the selected sequences) at a given or more rounds and is used for propose variants to the next round, as described in “Machine learning-assisted directed protein evolution with combinatorial libraries”, Z. Wu et al. arXiv preprint.

By Deep Sequencing is meant a repeated sequencing technique (hundreds or thousands of times) of a given region of DNA. This new generation sequencing approach allows the detection of rare clonal types, or cells of microorganisms whose genetic contribution can be of the order of 1% of the genetic material analyzed.

By Ultra Deep Sequencing techniques is meant a particular Deep Sequencing technique restricted to a limited region of the genome that allows the detection of variants whose percentage genetic contribution can be in the order of 10⁻⁷/10⁻⁸.

By genetic mutation is meant any stable and heritable modification in the nucleotide sequence of a genome or more generally of genetic material (both DNA and RNA) due to external agents or to chance, but not to genetic recombination.

By somatic mutation is meant a non-heritable genetic mutation.

By folding or protein folding, is meant the molecular folding process through which proteins reach their three-dimensional structure. By unfolded state of a protein is meant instead the denatured state of linear polypeptide chain.

By phenotype is meant the set of all the characteristics shown by a living organism, therefore its morphology, its development, its biochemical and physiological properties including behavior. By extension we refer to the phenotype of one or more mutations in a coding region of the genome meaning functional or structural variations

By genotype is meant the set of all the genes that make up the DNA (genetic makeup/genetic identity/genetic constitution) of an organism or population.

Epistasis generally means a non-additive phenotypic effect between individual mutations.

By residue is meant an amino acid of a protein or polypeptide

By molecular state is meant the state of the biological molecule (e.g., bound, non-bound, folded, unfolded), related to the activity or physical-chemical characteristic of the same;

By coding sequence is meant that portion of the DNA or RNA of a gene that codes for proteins.

By sequence alignment is meant a bioinformatics procedure in which two or more primary sequences of amino acids, DNA or RNA are arranged. in a matrix of common length by inserting appropriate symbols (not describing symbols related to amino acids or nitrogen bases) of insertion and deletion.

By positional bias is meant the frequency of observing a given amino acid in a specific position in a given sequence library or more generally in a multiple sequence alignment.

By phage display is meant a laboratory technique for the study of protein-protein, protein-peptide and protein-DNA interactions that uses bacteriophages (viruses that infect bacteria). With this technique, the gene coding for the protein of interest is inserted into the gene of a phage coating protein causing the exposure of the protein on the outside of the phage, keeping the gene of that protein inside, establishing a connection between genotype and phenotype.

By ribosome display is meant a biochemical technique to create proteins that bind a specific ligand, in particular, the technique consists in creating a hybrid between the protein of interest the progenitor RNA-messenger using as a complex to bind a particular ligand immobilized in different selection steps.

The present invention is directed to a computer implemented method for the analysis and use of sequence libraries deriving from screening experiments, e.g., Deep Mutational Scanning (DMS), Directed Evolution (DE) or SELEX, whose purpose is the selection or evaluation of amino acid or nucleotide sequences, which result in proteins and/or peptides or aptamers selected for a given chemical-physical characteristic during the screening experiment.

As described in the prior art, the DMS approach is aimed at selecting, given a desired characteristic, the best mutants starting from an initial pool of mutants and can be carried out using different types of experimental tests known in themselves.

The experiments can, for example, be based on cells (bacteria, yeasts and cultured mammalian cells) with a protein typically expressed by a plasmid or virus, or on the use of systems that develop in-vitro, such as the phage display or ribosome display.

In general, a library of mutated variants of the gene of interest is synthesized with the DMS, cloned in the appropriate expression vector and introduced, for example, in cells where the protein encoded by the gene has a function that can be selected. The selection can be applied for the protein function or another molecular property of interest, altering the frequency of each variant based on its functional capacity.

The selection in the screening experiment can be carried out using different strategies based on enzymatic catalysis or on binding to a molecular target, on cell growth favored by the presence of a more or less effective variant or on the separation of cells that express a specific variant. For example, selection can enrich cells with more active protein variants and exhaust those with inactive or highly inefficient variants. The selection can also be made by implementing the physical separation of the variants, such as in display experiments or by using cell separation techniques known in the art. The selection can finally be made before or after specific treatments or periods of time. In any case, the basis of the approach remains a selection process for an established characteristic.

At the end of one or more selection rounds, both the library present in the initial input population and that of the post-selection population are recovered and the frequency of each variant in the two libraries is determined by high-performance DNA sequencing techniques, in particular Deep Sequencing and Ultra Deep Sequencing.

As described in the prior art, SELEX's approach is aimed at selecting aptamers capable of binding with high specificity to a selected molecular target (proteins, other nucleic acids, entire cells, for example cancer). Also in this case, a library of oligonucleotide sequences is produced. Each sequence contains two constant regions at the ends to allow PCR amplification, and a central region generated with a random sequence of nucleotides.

These sequences are then subjected to an in vitro selection procedure in order to separate and amplify mainly functional aptamers rather than the other sequences. The selection can also be based in this case on different techniques known to those skilled in the art, for example on the binding affinity for a specific molecular target or on a catalytic activity. In any case, the basis of the approach remains a selection process for an established characteristic.

One or more rounds of amplification, selection and separation of the selected molecules can be carried out.

At the end of one or more selection rounds, the selected sequences are recovered and analyzed by means of high-performance DNA sequencing techniques, in particular Deep Sequencing.

Starting from the SELEX technique, various variants have been developed with different selection and amplification strategies known to those skilled in the art, such as described in “Zhuo Z, et al.—Recent Advances in SELEX Technology and Aptamer Applications in Biomedicine. Int J Mol Sci. 2017 Oct. 14; 18 (10)”.

The method according to the invention is therefore based on the use of a model capable of evaluating sequences that present one or more mutations with respect to the sequences of the training set, taking into consideration not only the possibility that these mutations may occur in different positions along the sequence but also, according to an embodiment example, their relative epistasis.

The selection takes place through a score that is based on a statistical energy function of the sequence in the method description section.

Once the model has been trained on a set of input sequences (a DMS experiment for example), the model can be used to evaluate any sequence that is aligned with the mutant library used in the experimental screening.

The model in a preferred embodiment described below considers two states, selected or unselected with which a probability is associated for each sequence.

However, it can be generalized to several states with which a probability is associated for each sequence. For example, in a preferred embodiment three states are considered: bound, unbound and folded, unfolded.

in the detailed description of the model, reference is made to the general version which considers a generic number of states and in particular the two-state case is described.

Notation

Underscored symbols as x refer to vectors whose elements denote sequences {xₛ}. Bold symbols x denote a set of distributed quantities over all the sequences and rounds, {xₛ(t)}, for each sequence s and iteration t.

Symbol Definitions

Rₛ(t) is the number of reads of sequence s in round t.

Nₛ(t) is the number of vectors transporting sequence s in round t.

$${N}_{tot} ( t ) = {\sum }_{𝓈} {N}_{𝓈} ( t )$$

is the total number of vectors transporting sequence s in round t.

$${R}_{tot} ( t ) = {\sum }_{𝓈} {R}_{𝓈} ( t )$$

is the total number of reads in round t.

$${n}_{tot} ( t ) = {\sum }_{𝓈} {\sum }_{k ∈ 𝓈 e l} {n}_{𝓈 , k} ( t )$$

is the total number of vectors with sequence s in a state k in round t.

n_{s,k}(t) is the number of vectors with sequence s in a state k in round t.

d is the number of distinct sequences.

Eₖ(s) is the statistic energy of sequence s in state k.

k∈sel is the set of molecular discrete states specifying the selection to a sequent round of the screening experiment from which training data come from (e.g., non-specific bond, bond, folded, unfolded).

In general, statistic energy is a linear multivariate function. In a first example, the energy of each state is defined as a linear function of energetic parameters θₖ₍ᵢ_{₁}_{, . . . , i}_{ₚ}₎(sᵢ_{₁}, . . . , sᵢ_{ₚ}) expressing both independent position bias and epistatic effects (i.e., non-additivity of mutational effects) such as the droplet, triplet interactions:

$${E}_{k} ( 𝓈 ) = - {\sum }_{i} {θ}_{ki} ( {𝓈}_{i} ) - {\sum }_{i < j} {θ}_{k ( ij )} ( {𝓈}_{i} , {𝓈}_{j} ) - {\sum }_{i < j < i} {θ}_{k ( ijl )} ( {𝓈}_{i} , {𝓈}_{j ,} {𝓈}_{l} ) + ⋯$$

Each θₖ₍ᵢ_{₁}_{, . . . , i}_{ₚ}₎(sᵢ_{₁}, . . . , sᵢ_{ₚ}) is the statistic energy contribution depending on p amino acids sᵢ_{₁}, . . . , sᵢ_{ₚ} having a position i₁, . . . , iₚ. All together, they build the free parameters that are calculated as scalars during the training procedure.

The expression above is not to be considered as exhaustive of the possible formulations of statistical energy. For example, statistical energy can be defined alternatively through the following multivariate linear function:

$${E}_{k} ( 𝓈 ) = - {U}_{k} ( L ) - {\sum }_{i = 1}^{L} {θ}_{k} ( {𝓈}_{i} ) - {\sum }_{j = 1}^{J} {\sum }_{i = 1}^{L - j} {θ}_{k ( j )} ( {𝓈}_{i} , {𝓈}_{i + j} )$$

This expression can be used, for example, without aligning the sequences in a multiple alignment (see definitions above).

In addition to the parameters θ dependent on the sequence, there is a term Uₖ that expresses an energy-statistical contribution dependent on the length L of the sequence.

It is also possible that the statistical energy, for example when the sequences are not particularly long, is defined only by the independent position bias, i.e., only the first term of the expression according to the first example above.

In general, the parameters are calculated for a predefined selection during a screening experiment of at least one chemical-physical characteristic of interest of the molecule.

T is the number of rounds/cycles

C is the number of targets.

[symbol] (n(t), C) is a function defined as follows: [symbol] (n(t), C)=1 if

$${\sum }_{𝓈 , k} {n}_{s , k} ( t ) ⩽ C and ( n ( t ) , C ) = 0$$

in the other case.

Referring to a screening experiment, likelihood is defined as the joint probability of T rounds of selection and amplification as follows (eq. 1):

$$( p , n , N , R ) = {}_{reg} ( p ) ( N ( 0 ) ) {\prod }_{t = 0}^{T - 1} ( R ( t ) \vert N ( t ) ) ( N ( t + 1 ) \vert n ( t ) ) ( n ( t ) \vert N ( t ) , p )$$

Where [symbol] _{reg}(p) is the regularization term and term [symbol] (N(0)) refers to the distribution of vectors present in round zero. Other three factors have the following definitions.

Read factor [symbol] (R(t)|N(t)) is the probability to extract a set of reads R={Rₛ(t)} from a distribution of vectors N={Nₛ(t)} and is defined by the flowing equation:

$$( R ( t ) \vert N ( t ) ) = \frac{{R}_{tot} ( t ) !}{{\prod }_{𝓈} {R}_{𝓈} ( t ) !} {\prod }_{𝓈 = 1}^{d} {( \frac{{N}_{𝓈} ( t )}{{N}_{tot} ( t )} )}^{{R}_{𝓈} ( t )}$$

where Rₜₒₜ(t) is the total number of reads in round t.

The second term is an amplification factor, i.e., the probability to have N(t+1) vectors amplified in round t+1 starting from n(t) vectors selected in round t, is defined as:

$$( N ( t + 1 ) \vert n ( t ) ) = \frac{{N}_{tot} ( t + 1 ) !}{{\prod }_{𝓈} {N}_{𝓈} ( t + 1 ) !} {\prod }_{𝓈 = 1}^{d} {( \frac{{Σ}_{k ∈ sel} {n}_{𝓈 , k} ( t )}{{n}_{tot} ( t )} )}^{{N}_{𝓈} ( t + 1 )}$$

The third term refers to selection and represents the probability to select n(t) vectors from N(t) present vectors and is defined as:

$$( n ( t ) \vert N ( t ) ) = \frac{( n ( t ) , C ) {\prod }_{𝓈} [ ( \begin{matrix}{N}_{𝓈} ( t ) \\ {n}_{k , 𝓈} ( t ) , …\end{matrix} ) {\prod }_{k} {e}^{- {n}_{𝓈 , k ( t ) {E}_{k} ( 𝓈 )}} ]}{{\sum }_{{n}^{′} ( t )} 𝒮 ( {n}^{′} ( t ) , C ) [ {\prod }_{k} {e}^{- {n}_{𝓈 , k ( t ) {E}_{k} ( 𝓈 )}} ]}$$

The learning or training procedure consists in finding, via known optimization algorithms, the scalar values of energy parameters θₖ₍ᵢ_{₁}_{, . . . , i}_{ₚ}₎(sᵢ_{₁}, . . . , sᵢ_{ₚ}) that maximize the a posteriori joint probability [symbol] and assigning to reads R={Rₛ(t)} training experimental data from a screening experiment.

An Example of a Preferred Embodiment: a Two-State System with a Rare and Deterministic Link and the Description of the Epistatic Effect with Two-Site Interactions.

In a preferred embodiment it is possible to consider only two states, i.e., selected and not selected (for example tied or not tied to the target).

Furthermore, it is assumed that the case of bound molecule is a rare phenomenon, therefore with a probability much less than 1.

A further assumption is an infinite number of targets C→∞, this approximation is realistic if the number of targets is much higher the number of vectors presented in the screening experiments. This condition is verified in the majority of experiments.

In such a case, state index k is eliminated because there exist k−1 statistical energies exist and, in such a case, the states are 2.

Let nₛ(t) be the number of vectors bonded with sequence s in round t.

According to such assumptions, the three factors described above are simplified as follows:

Reads factor [symbol] (R(t)|N(t)) is eliminated considering R(t)≈N(t)).

The amplification factor becomes:

$$( N ( t + 1 ) \vert n ( t ) ) = \frac{{N}_{tot} ( t + 1 ) !}{{\prod }_{𝓈} {N}_{𝓈} ( t + 1 ) !} {\prod }_{𝓈 = 1}^{d} {( \frac{{n}_{𝓈} ( t )}{{n}_{tot} ( t )} )}^{{N}_{𝓈} ( t + 1 )}$$

The selection factor becomes:

$$( n ( t ) \vert N ( t ) ) = {\prod }_{𝒮} [ ( \begin{matrix}{N}_{𝓈} ( t ) \\ {n}_{𝓈} ( t )\end{matrix} ) {{p}_{𝓈}^{{n}_{𝓈} ( t )} ( 1 - {p}_{𝓈} )}^{{N}_{𝓈} ( t ) - {n}_{𝓈} ( t )} ] ,$$

where pₛ is the probability that sequence s is selected and is defined as pₛ=1/(e^{E}^{ₛ}+1), where Eₛ=E_{bound}(s)−E_{unbound}(s).

Considering that pₛ<<1 it is possible to approximate pₛ≈e^{E}^{ₛ}.

In the current example, the energy is parametrized with interactions for one and two sites:

$${E}_{s} = - {\sum }_{i} {θ}_{i} ( {s}_{i} ) - {\sum }_{i < j} {θ}_{ij} ( {s}_{i} , {s}_{j} )$$

This expression, together with the probability of selecting a sequence, builds the genotype-phenotype map, in that it associates the sequence (genotype) with a probability of being in a molecular state (phenotype).

From this it follows that the logarithm of the joint probability becomes:

$$= {\sum }_{s , t} {N}_{s} ( t + 1 ) ln \frac{{N}_{s} ( t ) {e}^{- {E}_{s}}}{{\sum }_{r} {N}_{r} ( t ) {e}^{- {E}_{r}}}$$

The adopted approximations render the maximization of [symbol] a concave optimization problem for parameters θᵢ and θᵢⱼ. The aforementioned problem can be solved using the L-BFGS optimization algorithm once the sequences selected through the experiments have been assigned as N (t).

It can also be said that even the most general version of the problem that intends to calculate the parameters θᵢ and θᵢⱼ by maximizing the likelihood function in its more general shape, i.e. eq. 1, can be solved by numerical methods.

Input Data for Training.

The input data come from screening experiments of variants of biological polymer molecules, for example Deep Mutational Scan, Directed Evolution or techniques based on SELEX.

The input data are biological sequences, e.g., amino acids or nucleotides of the mutants used in the experiment together with the counts for the selection rounds.

These can be obtained from sequencing data (DNA reads in fastq format for example).

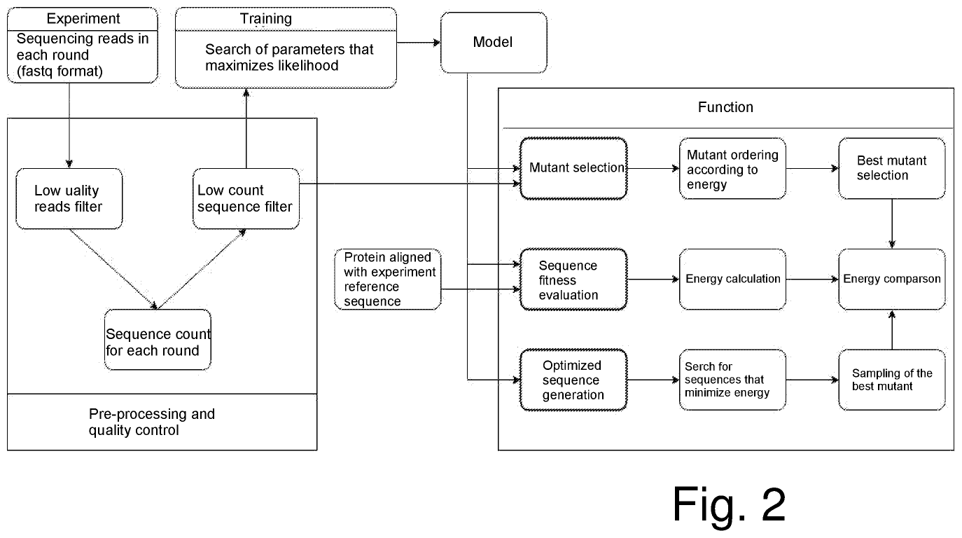

The typical bioinformatics procedure, described in FIG. 2, corresponds to the preprocessing carried out in the four datasets used as tests summarized in Table I below.

The sequencing consists of DNA filaments, starting from the set of reads for each round, for example in fastq format, and the procedure has the following steps:
filtering of reads with low sequencing quality or whose forward and reverse reads do not match;
translation of the nucleotide sequences in amino acid sequence, elimination of the sequences with a stop codon;
counting the number of sequences in each round;
filtering of sequences with a total number of occurrences in the various rounds of less than ten.

Training of the Probabilistic Model

This step, as indicated in the previous paragraphs, numerically solves the problem of maximizing the likelihood function as a function of the parameters of the at least one statistical energy function of the selection factor described above. The optimal values of the parameters thus obtained are characteristic of the set or set of training sequences entering the model and change if these training sequences are modified.

Uses of the Parameters of the Statistical Energy Function

The probabilistic method described above therefore analyzes sequencing data libraries deriving from experiments of selection and enrichment of mutant biological sequences with the aim of evaluating at least one input mutant biological sequence and selecting the best for an assigned chemical-physical characteristic. This characteristic is quantified, through the allowed molecular states, with a statistical energy function or with a combination of the statistical energies defined through the energy parameters of each of the statistical energy functions calculated during the learning phase.

Subsequently it is possible, starting from these parameters, to generate a library of biological sequences with the chemical-physical characteristic of interest.

The model is then applicable for:
Evaluating the mutants and selecting the best ones based on a given characteristic. In particular, once the energy parameters as defined above have been determined, it is possible to apply these parameters to calculate the at least one statistical energy function relating to an input sequence, as a non-limiting example to a new sequence i.e., not belonging to the sequences used in the training step. Statistical energies θ, once the relative energy parameters have been calculated by maximizing the likelihood function, are in fact functions whose unknown is a biological sequence. From the energy functions each of which is associated with the related molecular states observed during the selection experiment, it is possible to obtain the score of a sequence associated with the desired chemical-physical characteristic to which the molecular states refer.
Generating a library of biological sequences (e.g., of proteins with a given chemical-physical characteristic), and inferring a set of sequences characterized by an optimized function of statistical energy.

Mutant Evaluation.

In a preferred embodiment of the method, a score of a given biological sequence (amino acid or nucleotide) is calculated relative to a characteristic or activity of the related coded protein. The biological sequence to be evaluated can come from the training data (first row of the box on the right of FIG. 2) or from other experiments whose data have not been used for learning the model (second row of the box on the right of FIG. 2).

In the two-state construction (e.g., link present or absent) formulated above, the score is for example identified with the statistical energy itself:

$${E}_{s} = - {\sum }_{i} {θ}_{i} ( {s}_{i} ) - {\sum }_{i < j} {θ}_{ij} ( {s}_{i} , {s}_{j} )$$

In an embodiment with three states (e.g., bonded, non-bonded and non-folded states) score Φ can be defined as the following combination of energies of the bonded state E_{L} and the folded state E_{F}:

$$Φ ( s ) = \frac{1}{1 + {e}^{{E}_{L} ( s )} ( 1 + {e}^{{E}_{F} ( s )} )}$$

The calculation of the score is performed simply by selecting the parameters of the statistical energy relating to the sequence of the input mutant and applying the formula of the score, which in a two-state example case corresponds to the expression of the statistical energy function of the previous paragraph.

In particular, a high value of the previous three-state score is equivalent to a high probability that the related molecules are in the bonded and folded state in a given experiment. On the other hand, if the score is defined as identical to the statistical energy, a low value indicates the high probability of being in the bound state.

Generation One or More Optimal Sequences.

In a preferred embodiment of the method, it is possible to generate sequences that maximize the score function Φₛ of the model. The latter is defined in general starting from energy functions Eₖ(s) of each considered state k.

The generation of sequences with an optimal score takes place through a search algorithm of the sequence(s) that maximizes in an absolute or relative way an assigned score function Φₛ. An efficient algorithm for this is simulated annealing, a standard optimization algorithm.

According to a preferred embodiment, the data are derived from DMS experiments using one of the protein display techniques as described in (Fowler, Douglas M., and Stanley Fields. “Deep mutational scanning: a new style of protein science.” Nature methods 11.8 (2014): 801.).

According to another preferred embodiment, the data are derived from DMS and Directed Evolution (DE) experiments that are aimed at selecting effective protein variants in binding a specific molecular target, preferably a peptide or protein, and the method has the purpose of selecting the most selective protein variants among those analyzed.

According to another preferred embodiment, the data are derived from DMS and DE-experiments which are aimed at selecting protein variants effective in binding a specific molecular target, preferably a peptide or protein, and the method has the aim of generating a library of variants more selective proteins for the molecular target.

According to another preferred embodiment, the data are derived from DMS and DE experiments which are aimed at selecting protein variants effective in carrying out a specific catalysis and the method according to the invention has the purpose of generating a library of more active protein variants in said enzymatic catalysis.

According to another preferred embodiment, the data are derived from DMS and DE experiments which are aimed at selecting protein variants effective in carrying out a specific catalysis and the method according to the invention has the purpose of selecting from the library the most active protein variants in the enzymatic catalysis.

According to another preferred embodiment, the data are derived from DMS and DE experiments which are aimed at selecting protein variants effective in achieving optimal photoluminescence and the method according to the invention has the purpose of selecting the most photoluminescent variants from the library.

According to another preferred embodiment, the data are derived from DMS and DE experiments which are aimed at selecting active protein variants at high temperature and the method according to the invention has the purpose of selecting the most heat-resistant variants from the library.

According to a preferred embodiment, the data are derived from SELEX experiments or techniques based on it, known to those skilled in the art; as a non-limiting example, see “Zhuo Z, et al.—Recent Advances in SELEX Technology and Aptamer Applications in Biomedicine Int J Mol Sci. 2017 Oct. 14; 18 (10)”

According to another preferred embodiment, the data are derived from SELEX experiments or techniques based on it which are aimed at selecting aptamers effective in binding a specific molecular target and the method has the purpose of selecting the most selective aptamers among those analyzed.

According to another preferred embodiment, the data are derived from SELEX experiments or techniques based on it which are aimed at selecting aptamers effective in binding a specific molecular target and the method aims to generate a library of more selective aptamer variants for the molecular target.

According to another preferred embodiment, the method is applied to the so-called “Machine-learning-assisted Directed Evolution” process. In this embodiment, the method is therefore trained on data derived from one or more Direct Evolution rounds and applied to generate effective protein variants to be changed and tested in subsequent Direct Evolution rounds, according to the scheme called Machine-Learning-assisted Directed Evolution.

The method of the present invention is therefore effective and reliable for the in-silico screening of libraries of protein or nucleotide sequences obtained from experiments of DMS, DE or techniques based on SELEX and for the selection of mutants with the desired characteristics. According to the method of the present invention, it is also possible to use data derived from these experiments to obtain a library of high efficiency sequences of a chemical-physical characteristic of a molecule whereby high efficiency is meant, for example, high catalysis capacity, high fitness, high ability to bind to a specific molecular target.

The method according to the invention is generally applicable to all types of DMS or DE experiments with at least one selection cycle.

The method according to the invention is generally applicable to all types of HTS-SELEX (High-Throughput Sequencing SELEX) experiments and techniques derived from it with at least one selection cycle.

### EXAMPLES

The following examples are illustrative of the invention and are in no case to be considered limitative of the relative scope.

We report below the performance of the method with data deriving from four DMS experiments briefly described below and whose characteristics are summarized in the table below.

TABLE I

| | | | # | # | |
|---|---|---|---|---|---|
| | | Selection | mutated | sequenced | # |
| Bibliography | Protein | target | positions | roundsi | mutants |
| Boyer et al | Antibody | PVP, DNA | 4 | 3 | ~ |
| PNAS (2016) | heavy chain. | | | | 2.5 × 10⁴ |
| Fowler et al. | hYAP65 | GTPPPPYTVG | 25 | 3 | ~ |
| Nature methods | (WW | peptide | | | 3 × 10⁵ |
| (2010) | domain) | | | | |
| Araya et al. | hYAP65 | GTPPPPYTVG | 34 | 4 | ~ |
| PNAS (2012) | (WW | peptide | | | 6 × 10⁵ |
| | domain) | | | | |
| Olson et al. | GB1. | IgG-Fc | 55 | 2 | ~ |
| Current Biology | | | | | 5.3 × 10⁵ |
| (2014) | | | | | |

### Example 1

### Prediction of the Selectivity of Binding of Mutant Antibodies on Data Deriving from a DMS Experiment Carried Out by Means of a Phage Display

The model has been tested on data published by S. Boyer et al, “Hierarchy and extremes in selections from pools of randomized proteins.” PNAS (2016).

The DMS experiment reported is aimed at the analysis of a library of antibodies and the bond with a neutral synthetic polymer, polyvinylpyrrolidone (PVP). In this case, the experiment was conducted by carrying out three rounds of amplification and selection using the phage display technique. The initial library was created by saturation mutagenesis of an anti-PVP antibody on four consecutive amino acids of the region that determines complementarity 3 (CDR3).

### Example 2

### Prediction of the Binding Selectivity of WW Domain Mutants of the hYAP65 Protein, Data Deriving from a DMS Experiment Carried Out Using a Phage Display

The model has been tested on data published by D. M. Fowler et al, “High-resolution mapping of protein sequence-function relationships.” Nature methods (2010).

The DMS experiment reported is aimed at analyzing a library of WW domain mutants selected to bind a peptide ligand (GTPPPPYTVG). The experiment was conducted by carrying out 6 rounds of amplification and selection using the phage display technique and sequencing 3 rounds (0.3,6). The initial library was created with the “doped oligonucleotide synthesis” technique.

### Example 3

### Prediction of the Binding Selectivity of WW Domain Mutants of the hYAP65 Protein, Data Deriving from a DMS Experiment Carried Out by Means of Phage Display

The model has been tested on data published by C. L. Araya et al. “A fundamental protein property, thermodynamic stability, revealed solely from large-scale measurements of protein function.” PNAS (2012).

The DMS experiment reported is aimed at analyzing a library of WW domain mutants selected to bind a peptide ligand. In this case, the experiment was conducted by carrying out 4 rounds of amplification and selection using the phage display technique. The initial library was created through chemical synthesis of DNA with “doped nucleotide pools”.

### Example 4

### Prediction of the Binding Selectivity of Mutants of the Binding Domain with the Immunoglobulin G (IgG) of the G Protein (IgG-Binding Domain of Protein G) (GB1), Data Deriving from a DMS Experiment Performed by Means of mRNA Display

The model has been tested on data published by C. Olson et al. “A comprehensive biophysical description of pairwise epistasis throughout an entire protein domain.” Current Biology (2014).

The DMS experiment reported is aimed at analyzing a library of mutants of the GB1 protein selected in the binding with IgG-FC. The experiment was conducted, in this case, by carrying out an amplification and selection round using the mRNA display. The initial library was created with a saturation-mutagenesis technique.

Type of Test

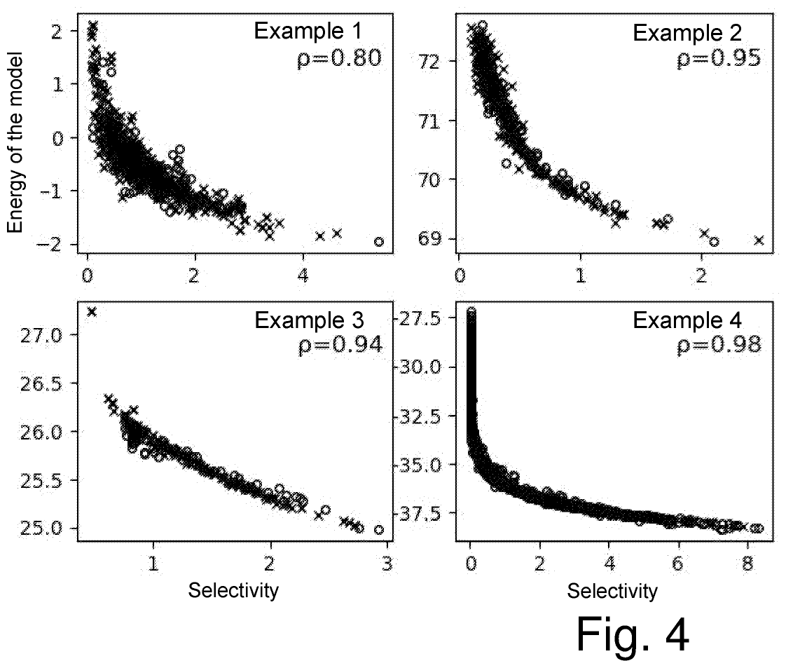

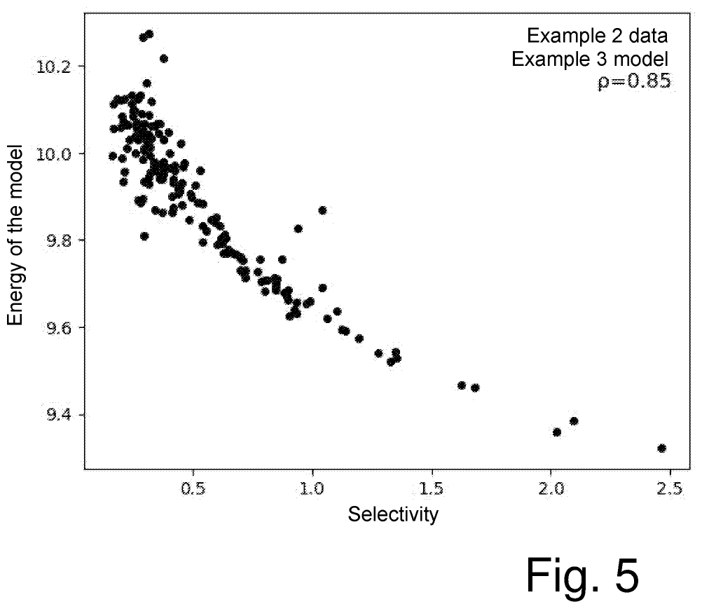

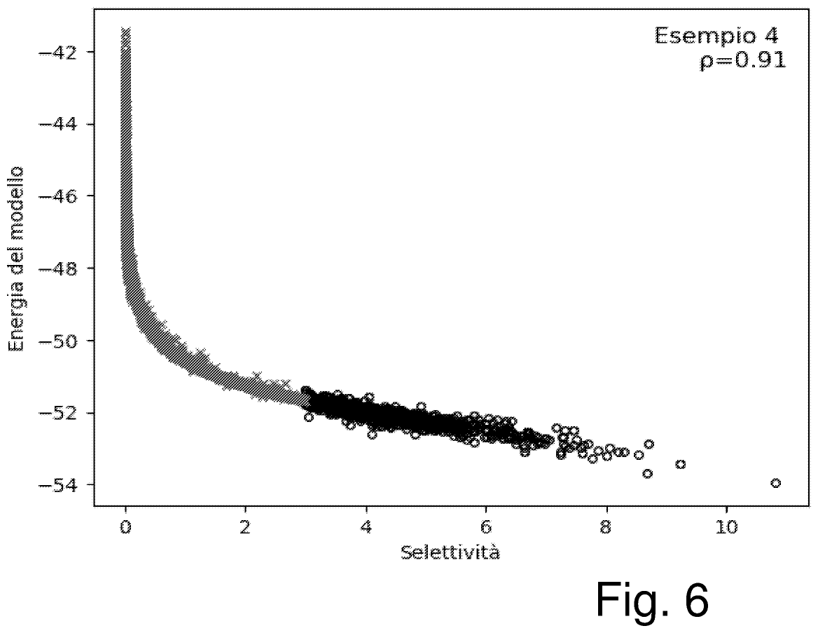

The dataset derived from these experiments was divided randomly into a training set for the model and a test set in which the statistical binding energy of the model was compared with an experimental measure of the ability of the mutants to bind and then be selected for the next round. This measure of a mutant's selectivity is defined as the ratio of the frequency of occurrence of the sequence in one round with the next one averaged over successive pairs of rounds. In formulas we define the selectivity of a sequence s as:

$${Sel}_{s} = {\sum }_{t = 1}^{T - 1} {f}_{s} ( t + 1 ) / {f}_{s} ( t )$$

Where fₛ(t) is the frequency of the sequence s under examination at round t and T the total number of rounds (FIGS. 4, 5 and 6).

The embodiment of the method is reported in the previous paragraphs relating to a two-state system with rare and deterministic bond and the description of the epistatic effect with two-site interactions.

What is claimed is:

1. A computer-based method for processing results of a biological sequence screening experiment, comprising the steps of:
a) receiving a set of sample biological sequences selected by at least one screening experiment comprising a selection step, wherein in the selection step, molecules encoded by the sample biological sequences are selected based on a chemical-physical property of interest;
b) defining allowed molecular states related to the chemical-physical property of interest and at least one statistical energy function, wherein the at least one statistical enemy function is expressed in terms of a multivariate linear function of specific statistical energy parameters and associated to the allowed molecular states;
c) providing an expression of a likelihood of obtaining, from the at least one screening experiment, the set of sample biological sequences observed in different rounds of the at least one screening experiment, wherein the expression comprises a selection factor expressing a probability that a sequence is selected during the at least one screening experiment as a function of the specific statistical energy parameters;
d) calculating energy parameters of the multivariate linear function by maximizing the expression of the likelihood and considering the set of sample biological sequences; and
e) given at least one input sequence, calculating a score of the at least one input sequence based on the at least one statistical energy function identified by the energy parameters calculated in step d to represent an evaluation of the at least one input sequence with respect to the chemical-physical property of interest; and/or
generating at least one sequence that maximizes at least locally a score function based on the at least one statistical energy function identified by the energy parameters calculated in step d.

2. The computer-based method according to claim 1, wherein in step d, the set of sample biological sequences are used to define unsupervised training avoiding experimental measurements of physical or chemical parameters.

3. The computer-based method according to claim 1, wherein the expression of the likelihood further comprises an amplification factor and/or a sampling factor, wherein the amplification factor is expressed by a probability that a sequence is amplified during the at least one screening experiment, and the sampling factor is expressed by a probability that a sequence is sampled during the at least one screening experiment.

4. The computer-based method according to claim 1, wherein the selection factor is
$$( \underline{n} ( t ) \vert \underline{N} ( t ) ) = \frac{( \underline{n} ( t ) , C ) {\prod }_{s} [ ( \begin{matrix}{N}_{s} ( t ) \\ {n}_{k , s} ( t ) , …\end{matrix} ) {\prod }_{k} {e}^{- {n}_{s , k} ( t ) {E}_{k} ( s )} ]}{{\sum }_{{\underline{n}}^{′} ( t )} ( {\underline{n}}^{′} ( t ) , C ) {\prod }_{s} ( \begin{matrix}{N}_{s} ( t ) \\ {n}_{k , s} ( t ) , …\end{matrix} ) [ {\prod }_{k} {e}^{- {n}_{s , k} ( t ) {E}_{k} ( s )} ]} ,$$
wherein:
Rₛ(t) is a number of reads of sequence s in round t,
Nₛ(t) is a number of biological vectors that transfer the sequence s at the round t,
$${N}_{tot} ( t ) = {\sum }_{s} {N}_{s} ( t )$$
is a total number of the biological vectors transferring the sequence s in the round t,
$${R}_{tot} ( t ) = {\sum }_{s} {R}_{s} ( t )$$
is a total number of reads in the round i,
$${n}_{tot} ( t ) = {\sum }_{s} {\sum }_{k ∈ sel} {n}_{s , k} ( t )$$
is a total number of biological vectors with the sequence s in state k in the round t,
wherein,
n_{s,k}(t) is a number of the biological vectors with the sequence s in the state k in the round t,
d is a number of different sequences,
Eₖ(s) is a statistic energy of the sequence s in the state k,
k∈sel is a set of discrete molecular states that describe the selection step to a following round of the at least one screening experiment, wherein training data are from the set of discrete molecular states, and the set of discrete molecular states comprises non-specific bond, specific bond, folded state, and unfolded state,
C is a target number, [symbol]
(n(t), C) is a function defined as follows: [symbol] (n(t), C)=1 if
$${\sum }_{s , k} {n}_{s , k} ( t ) ⩽ C , or ( \underline{n} ( t ) , C ) = 0$$
in any other case.

5. The computer-based method according to claim 1, wherein the at least one statistical energy function comprises terms expressing independent position biases and epistatic effects.

6. The computer-based method according to claim 5, wherein the at least one statistical energy function is expressed by:
$${E}_{k} ( s ) = - {\sum }_{i} {θ}_{ki} ( {s}_{i} ) - {\sum }_{i < j} {θ}_{k ( ij )} ( {s}_{i} , {s}_{j} ) - {\sum }_{i < j < l} {θ}_{k ( ijl )} ( {s}_{i} , {s}_{j} , {s}_{l} ) + … .$$

7. The computer-based method according to claim 5, wherein the at least one statistical energy function is expressed by:
$${E}_{k} ( s ) = - {U}_{k} ( L ) - {\sum }_{i = 1}^{L} {θ}_{k} ( {s}_{i} ) - {\sum }_{j = 1}^{J} {\sum }_{i = 1}^{L - j} {θ}_{k ( j )} ( {s}_{i} , {s}_{i + j} )$$

8. The computer-based method according to claim 4, wherein the expression of the likelihood comprises an amplification factor expressed by:
$$= ( \underline{N} ( t + 1 ) \vert \underline{n} ( t ) ) = \frac{{N}_{tot} ( t + 1 ) !}{{\prod }_{s} {N}_{s} ( t + 1 ) !} {\prod }_{s = 1}^{d} {( \frac{{\sum }_{k ∈ sel} {n}_{s , k} ( t )}{{n}_{tot} ( t )} )}^{{N}_{s} ( t + 1 )} .$$

9. The computer-based method according to claim 8, wherein the expression of the likelihood comprises a reads sampling factor expressed by:
$$( \underline{R} ( t ) \vert \underline{n} ( t ) ) = \frac{{R}_{tot} ( t ) !}{{\prod }_{s} {R}_{s} ( t ) !} {\prod }_{s = 1}^{d} {( \frac{{N}_{s} ( t )}{{N}_{tot} ( t )} )}^{{R}_{s} ( t )} .$$

10. The computer-based method according to claim 9, wherein a number of the allowed molecular states is two, and the allowed molecular states are selected or not-selected in the at least one screening experiment.

11. The computer-based method according to claim 10, wherein the selection factor is expressed by:
$$( \underline{n} ( t ) \vert \underline{N} ( t ) ) = {\prod }_{s} [ ( \begin{matrix}{N}_{s} ( t ) \\ {n}_{s} ( t )\end{matrix} ) {{p}_{a}^{{n}_{s} ( t )} ( 1 - {p}_{s} )}^{{N}_{s} ( t ) - {n}_{s} ( t )} ] .$$
